(19) United States
(12) Patent Application Publication
(10) Pub. No.: US 2026/0199202 A1
(43) Pub. Date: Jul. 16, 2026

(54) MATTIFYING COSMETIC COMPOSITION COMPRISING AT LEAST A CLAY AND A CRYSTALLIZED FATTY PHASE

(71) Applicant: L'OREAL, Paris (FR)

(72) Inventors: Céline PHILIPPON, Chevilly Larue (FR); Franck CLEMENT, Chevilly Larue (FR); Katia PERRIN, Chevilly Larue (FR); Catherine MARION, Chevilly Larue (FR)

(73) Assignee: L'OREAL, Paris (FR)

(21) Appl. No.: 19/138,295

(22) PCT Filed: Dec. 21, 2023
(86) PCT No.: PCT/EP2023/087466

§ 371 Date: Jun. 12, 2025

(30) Foreign Application Priority Data
Dec. 23, 2022 (FR) FR2214393

(51) Int. Cl. A61K 8/25; A61K 8/36; A61Q 1/12

(52) U.S. Cl. CPC A61K 8/25; A61K 8/361; A61Q 1/12; A61K 2800/412; A61K 2800/652

(57) ABSTRACT

The present invention relates to compositions, in particular cosmetic compositions, comprising, in a physiologically acceptable aqueous medium, at least one clay chosen from phyllosilicates having a 2:1 sheet structure and at least one crystallizable fatty substance in crystallized form. The present invention also relates to the use of such a composition for regulating excess sebum of the skin and/or mattifying the skin and/or blurring skin imperfections.

The present invention relates to compositions, in particular cosmetic compositions, comprising, in a physiologically acceptable aqueous medium, at least one clay chosen from phyllosilicates having a 2:1 sheet structure and at least one crystallizable fatty substance in crystallized form. The present invention also relates to the use of such a composition for regulating excess sebum of the skin and/or mattifying the skin and/or blurring skin imperfections.

Shiny skin, generally linked to a substantial secretion of sebum, is a problem that affects adolescents, but which can also occur in adulthood under the effect in particular of a hyper-production of androgens or external factors such as pollution. Shiny skin can also be linked to sweat, resulting from physical activity or climate conditions.

Obtaining a skin mattifying effect is highly sought by users, because the reflections caused by excess sebum on the skin surface are generally considered as unattractive. Moreover, it is also known that shiny skin generally results in poorer stability of makeup which thus tends to degrade during the course of the day.

Galenic formulations intended for skin care and more particularly for regulating shine phenomena have therefore already been proposed. As a general rule, they comprise a so-called seboregulating agent, i.e., capable of helping regulate the activity of the sebaceous glands via an action which can be described as biological and/or one or more fillers absorbing sebum. Another effective means of rapidly reducing unsightly shiny areas consists of using “soft-focus” blurring-effect fillers. However, these fillers can give discomfort in particular fluff on the skin and/or a perception of unclean skin.

It is also known that clays make it possible to absorb excess sebum and regulate its production. For example, gels comprising hectorite in water exhibit substantial sweat and sebum absorption effectiveness. However, these gels are absolutely not stable (they exhibit a syneresis phenomenon very quickly after gel formation). Furthermore, the sensory properties of these gels are not acceptable, in particular because they give a rough skin finish.

There remains therefore a need for cosmetic compositions comprising at least one clay, which are stable, which provide a mattifying effect and/or excess sebum regulating and/or imperfection blurring effect that are effective while having satisfactory cosmetic properties such as a texture that is slick on application, non-tacky with a clean and non-rough skin finish.

The inventors have surprisingly discovered that associating a clay chosen from phyllosilicates having a 2:1 sheet structure and at least one crystallizable fatty substance in crystallized form makes it possible to obtain such a composition.

The present invention therefore relates to a composition, in particular a cosmetic composition, comprising, in a physiologically acceptable aqueous medium:
at least one clay chosen from phyllosilicates having a 2:1 sheet structure, and
at least one crystallizable fatty substance in crystallized form.

The present invention also relates to the cosmetic use of a composition according to the invention for regulating excess sebum of the skin and/or mattifying the skin and/or blurring skin imperfections.

The present invention also relates to a cosmetic method for regulating excess sebum of the skin and/or for mattifying the skin and/or blurring skin imperfections, characterized in that it consists of applying on the skin at least one cosmetic composition according to the invention.

The present invention furthermore relates to the use of at least one crystallizable fatty substance in crystallized form in a cosmetic composition comprising at least one clay chosen from phyllosilicates having a 2:1 sheet structure as defined in the present description, for enhancing the mattifying and/or blurring effect of said cosmetic composition.

Clay Chosen from Phyllosilicates Having a 2:1 Sheet Structure

The composition according to the invention comprises at least one clay chosen from phyllosilicates having a 2:1 sheet structure. The classification of clays is referenced in the following book: Bergaya F., Lagaly G., “Handbook of Clay Science,” 2nd Edition. A. Fundamentals—Elsevier Ltd., 2013.

Phyllosilicates are minerals in the silicate group made by stacking tetrahedral (“T”) layers in which the tetrahedrons share three out of four vertices (the “basal” oxygens), with the fourth vertex (the “apical” oxygen) being linked to an octahedral (“O”) layer occupied by different cations. In phyllosilicates having a TOT structure, an octahedral layer O is placed between two tetrahedral layers T.

The 2:1 sheet structure is also called a TOT structure, with T being a tetrahedral layer and O being an octahedral layer.

Preferably, the clay is chosen from smectites.

The structure of smectites differs from other phyllosilicates having a 2:1 sheet structure in the presence of an interfoliar space between each combination of TOT sheets, which depends on the hydration state of the clay, and in which interfoliar cations are placed in between. Smectites are sometimes called “swelling” clays. The interfoliar cations are preferably chosen from Ca²⁺, Mg²⁺, Na⁺, and Li⁺.

In particular, the clay can be chosen from dioctahedral or trioctahedral smectites.

Preferably, the clay is chosen from trioctahedral smectites.

Trioctahedral smectites are characterized in that all the octahedral sites of the TOT structure are primarily occupied by divalent cations. The divalent cations of the octahedral sites are preferably chosen from Mg²⁺ and Fe²⁺.

In particular, the clays according to the invention are trioctahedral smectites which can be exfoliated or “activated” in the presence of water, thus forming aqueous gels due to the existence of interfoliar cations within the structure.

When water comes into contact with a clay suitable for the invention, it hydrates the sheets of the clay, thus causing the distance between the sheets to swell. A separation of the sheets then possibly occurs through a mechanism of exfoliation and/or delamination. An aqueous gel is then obtained. Essentially, a content ranging from 0.5% to 10% by weight, in relation to the total weight of the composition, of this specific type of clay makes it possible to obtain gelified aqueous phases in the compositions according to the invention.

Preferably, the clay according to the invention comprises a quantity of SiO₂ by weight greater than or equal to 30%, in relation to the total weight of the clay, preferably between 35% and 65% by weight, and a quantity of MgO by weight greater than or equal to 10%, preferably between 15% and 30% by weight.

According to a more preferred embodiment, the clay according to the invention comprises 35% to 65% SiO₂ by weight, in relation to the total weight of the clay, and 15% to 30% MgO by weight, in relation to the total weight of the clay.

Preferably, in the clay according to the invention, the weight ratio of SiO₂ to MgO is between 1 and 3, preferably between 1.5 and 2.5, and preferentially between 1.8 and 2.4.

Preferably, the clay according to the invention further comprises a quantity by weight of Al₂O₃ between 0% and 15% in relation to the total weight of the clay.

According to a first variant, a clay suitable for the invention is free from (without) Al₂O₃.

According to a second variant, a clay suitable for the invention may comprise from 0.1% to 15% Al₂O₃ by weight, preferably 8% to 12% by weight, in relation to the total weight of the clay.

According to another preferred embodiment, when the Al₂O₃ is present in the clay suitable for the invention, the weight ratio of SiO₂ to Al₂O₃ is strictly greater than 3.

Preferably, the clay according to the invention has a density of less than 2.7. The density (or specific gravity) is a very important property of minerals; calculating it is quite simple. It does not have a unit and is measured by various devices and techniques which are covered in most mineralogy manuals. Among these methods, the Jolly balance and the beam balance are best suited for work on mineral samples. The device for taking these measurements is simple and can be built at little cost (Sinkankas, 1966). The density results from the following calculation:

$$Densi \text{?} é = ( poids dans l ’ air ) / ( poids dans \text{?} ’ air - poids dans \text{?} ’ eau ) .$$

$$\text{?} \text{indicates text missing or illegible when filed}$$

According to a particular embodiment, a clay suitable for the invention has the following general molecular formula:

$$[ \begin{matrix}{M}_{8 - x}^{4 +} & {{M}_{x}^{3 +} ]}^{tetra} [ \begin{matrix}{M}_{6 - y}^{2 +} & {{M}_{y}^{+} ]}^{octa} {{O}_{20} ( OH )}_{4} {X}_{\frac{x + y}{k}}^{k +}\end{matrix}\end{matrix} , n {H}_{2} O$$

wherein
M⁴⁺ is a cation, preferably Si⁴⁺,
M³⁺ is a cation, preferably Al³⁺,
M²⁺ is a cation, preferably Fe²⁺ or Mg²⁺,
M⁺ is a cation, preferably Li⁺,
X is an interfoliar cation, preferably Ca²⁺, Na⁺, Li⁺, or mixtures thereof,
x is the tetrahedral substitution rate,
y is the octahedral substitution rate,
k is the valence of the interfoliar cation X,
n is an integer, preferably from 0 to 100.

The clay suitable for the invention is generally available in powder form.

Preferably, the clay is chosen from the group consisting of hectorite, stevensite, saponite, sauconite, monomorillonite, beidellite, nontronite, ferrosaponite and saliotite. Advantageously, the clay is hectorite.

The hectorite can be defined by the following formula: Naₓ(Mg₃₋ₓ, Liₓ) Si₄O₁₀(OH)₂, with x being between 0.2 and 0.4, preferably equal to 0.3.

Preferably, the clay according to the invention is an unmodified clay. An unmodified clay means a natural or synthetic clay that has not undergone any modification of any kind. For example, when an unmodified clay suitable for the invention is an unmodified hectorite, this clay is different from the hectorites modified by a fatty-acid ammonium chloride at C₁₀ to C₂₂, such as hectorite modified by distearyl dimethyl ammonium chloride. According to one embodiment, an unmodified clay suitable for the invention is synthetic.

According to a preferred embodiment, an unmodified clay suitable for the invention is natural, preferably natural hectorite.

A modified clay refers to silicates in sheets in which the lipophilicity is increased, for example by ion-exchange reactions with quaternary ammonium salts, preferably quaternary ammonium chlorides. The quaternary ammonium chlorides are, for example, benzyldimethylstearylammonium chloride, or quaternium-18, with the formula

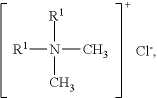

wherein the R1 groups are selected independently from the linear alkyl groups saturated at C12-C20. Modified clays are, for example, disteardimonium hectorite or stearalkonium hectorite.

The composition according to the invention is preferably substantially free from modified clays, preferably substantially free from disteardimonium hectorite and stearalkonium hectorite. “Substantially free” means that the composition comprises less than 0.1% by weight, in relation to the total weight of the composition, preferably less than 0.05% by weight, preferably less than 0.01% by weight of modified clay. Preferably, the composition according to the invention is completely free of modified clay.

The clay marketed by Elementis under the name Bentone EW or Hydroclay 2000 LO may be used as an unmodified clay and more particularly an unmodified hectorite.

Preferably, the clay is present with a content of between 0.5% and 8% by weight in relation to the total weight of the composition, preferably between 1% and 5% by weight, preferably between 1.5% and 3.5% by weight, advantageously between 2% and 3% by weight.

### Crystallizable Fatty Substance

The composition according to the invention comprises at least one crystallizable fatty substance in crystallized form.

The crystallizable fatty substance(s) in crystallized form are preferably crystallized in the form of spherulites. These spherulites are visible for example with optical microscopy with an X40 magnification, with DIC (Differential Interference Contrast). A spherulite is a structure in sphere or fan form, formed from at least one crystallizable fatty substance during the cooling of the composition, and organized as fluid or crystallized lamellar phases. Spherulites are also visible in polarized light. The crystallizable fatty substance(s) in crystallized form are therefore preferably crystallized in the form of spherulites organized as crystallized lamellar phases. The composition according to the invention can also comprise the fatty substance(s) crystallized in the form of spherulites organized as fluid lamellar phases.

Crystallizable fatty substance denotes according to the present invention a solid lipophilic compound deformable, or non-deformable at ambient temperature (25° C.) and having a melting point greater than or equal to 25° C., preferably between 25° C. and 200° C., preferably between 25° C. and 120° C.

According to the invention, the melting point is equivalent to the temperature of the most endothermic peak observed in thermal analysis (DSC) as described in the standard ISO 11357-3; 1999. The melting point of the crystallizable fatty substance may be measured using a differential scanning calorimeter (DSC), for example the calorimeter sold under the name MDSC 2920 by TA Instruments. Such a measurement method is for example described in the document PCT/EP2013/062964.

The crystallizable fatty substance(s) can be chosen from long-chain crystallizable fatty alcohols and mixtures thereof, preferably chosen from saturated or unsaturated fatty chain alcohols at C12-C24, preferably at C14-C20, preferably at C14-C18, for example from cetearyl alcohol (C16/C18 50/50), stearyl alcohol, myristyl alcohol, cetyl alcohol, C16-C22 alcohols.

The crystallizable fatty substance(s) can also be chosen from long-chain crystallizable esters and mixtures thereof, such as the INCI compound “CETYL ESTERS (and) CETYL ESTERS MYRISTYL STEARATE AND MYRISTYL PALMITATE MIXTURE”, or the INCI compound “MYRISTYL STEARATE AND MYRISTYL PALMITATE MIXTURE”, glycol distearate, glycol stearate, cetyl palmitate such as the commercial product ERCAWAX CP V/O from the supplier ERCA, isopropyl palmitate, C20-C40 alkyl stearates, long-chain crystallizable esters and mixtures thereof, such as for example the compound sold under the trade name COMPRITOL 888 CG ATO from Gattefosse (INCI: GLYCERYL DIBEHENATE (and) TRIBEHENIN (and) GLYCERYL BEHENATE) or each of its components taken separately, tricaprin, trilaurin, trimyristin, tripalmitin, tristearin, glycerol distearate, glyceryl distearate, glyceryl dipalmitostearate and linoleoyl polyoxyl-6 glyceride.

Preferably, the crystallizable esters are chosen from esters of saturated or unsaturated fatty acids at C10-C24, preferably at C12-C24, preferably at C14-C18, and saturated or unsaturated fatty chain alcohols, at C12-C24, preferably at C14-C20, preferably at C14-C18, such as the INCI compound “CETYL ESTERS” comprising a mixture of palmityl, cetyl and myristyl esters of palmitic, myristic and steric acids.

The crystallizable fatty substance(s) can also be chosen from long-chain crystallizable fatty acids and mixtures thereof, such as for example the INCI compound “STEARIC ACID”, mixtures of stearic acid and palmitic acid, saturated fatty acids at C4-C28 and unsaturated fatty acids at C4-C28. Fatty acids at C4-C28 include hydroxylated fatty acids at C4-C28.

The crystallizable fatty acids are preferably chosen from saturated or unsaturated fatty acids at C10-C24 and mixtures thereof, preferably at C12-C24, preferably at C14-C18, for example from myristic acid, palmitic acid and stearic acid. The fatty acid is optionally hydroxylated, for example can be hydroxystearic acid.

Some crystallizable fatty substances are commonly referred to as waxes.

Among the crystallizable fatty substances of mineral origin, mention can be made of: paraffin wax, ozokerite, ceresin and microcrystalline wax.

Among the crystallizable fatty substances of plant origin, mention can be made of: carnauba wax, candelilla wax such as that sold under the reference SP 75 G by Strahl & Pitsch, bay leaf wax, sugar cane wax, ceramide, alfa wax, olive tree wax, rice wax such as that sold under the reference NC 1720 by Cera Rica Noda, sunflower seed wax such as that sold by Koster Keunen under the reference sunflower wax, hydrogenated jojoba wax, hydrogenated castor oil, hydrogenated olive oil, hydrogenated cotton oil, Polyglyceryl-3 esters of green mimosa, jojoba and sunflower waxes, and absolute flower waxes such as essential blackcurrant flower wax, soy wax, myrica fruit wax or bay leaf wax.

Among the crystallizable fatty substances of animal origin, mention can be made of: beeswaxes or modified beeswaxes (cerabellina), lanolin and spermaceti.

The other crystallizable fatty substances which can be used according to the invention are in particular marine waxes, polyethylene waxes or polyolefin waxes in general, such as olefin α-oligomers, for example the Performa VR 825, 103 and 260 polymers sold by New Phase Technologies, ethylene/propylene copolymers, such as Performalene® EP 700, or Fischer-Tropsch waxes or a mixture of these products.

Preferably, the crystallizable fatty substance(s) is (are) chosen from long-chain crystallizable fatty alcohols and mixtures thereof, long-chain crystallizable esters and mixtures thereof and long-chain crystallizable fatty acids and mixtures thereof.

Preferably, the crystallizable fatty substance comprises at least a first crystallizable fatty substance, at least a second crystallizable fatty substance and at least a third crystallizable fatty substance, the first, second and third crystallizable fatty substance being different from each other, and the first, second and third crystallizable fatty substances being each in crystallized form.

Preferably, the first crystallizable fatty substance is chosen from saturated or unsaturated fatty chain alcohols at C12-C24, preferably at C14-C20, preferably at C14-C18. Advantageously, the first crystallizable fatty substance comprises a mixture of stearyl alcohol and cetyl alcohol, preferably cetearyl alcohol (50/50 mixture by weight of stearyl alcohol and cetyl alcohol).

Preferably, the first crystallizable fatty substance(s) is (are) present with a content of between 0.2% and 6% by weight in relation to the total weight of the composition, preferably between 0.5% and 5% by weight, preferably between 1% and 4% by weight, advantageously between 1% and 3% by weight.

Preferably, the second crystallizable fatty substance is chosen from saturated or unsaturated fatty chain esters, preferably chosen from esters of saturated or unsaturated fatty acid at C10-C24, preferably at C12-C24, preferably at C14-C18, and saturated or unsaturated fatty chain alcohols, at C12-C24, preferably at C14-C20, preferably at C14-C18, for example a mixture of at least two esters chosen from esters of palmityl, cetyl or myristyl alcohols, and palmitic, myristic or stearic acid, for example a mixture of myristyl stearate and myristyl palmitate.

Preferably, the second crystallizable fatty substance comprises a first fatty chain ester and a second fatty chain ester, as defined above. The first and second fatty chain esters are preferably esters of saturated or unsaturated fatty chain alcohol, at C12-C24, preferably at C14-C20, preferably at C14-C18, advantageously of myristic alcohol. The first fatty chain ester is preferably myristyl stearate. The second fatty chain ester is preferably myristyl palmitate. Preferably, the first fatty chain ester content is between 0.05% and 1% by weight in relation to the total weight of the composition, preferably between 0.1% and 0.5% by weight. Preferably, the second fatty chain ester content is between 0.05% and 0.8% by weight in relation to the total weight of the composition, preferably between 0.1% and 0.4% by weight.

Preferably, the second crystallizable fatty substance(s) is (are) present with a content of between 0.1% and 3% by weight in relation to the total weight of the composition, preferably between 0.2% and 2% by weight, preferably between 0.3% and 1% by weight, advantageously between 0.4% and 0.8% by weight.

Preferably, the third crystallizable fatty substance is chosen from saturated or unsaturated fatty acids at C10-C24 and mixtures thereof, preferably at C12-C24, preferably at C14-C18, for example from myristic acid, palmitic acid and stearic acid. Preferably, the third crystallizable fatty substance comprises a mixture of three saturated or unsaturated fatty acids at C10-C24 and mixtures thereof, preferably at C12-C24, preferably at C14-C18, for example a mixture of myristic acid, palmitic acid and stearic acid.

For example, the mass ratio between the content by weight of first fatty acid, preferably C18 fatty acid, preferably stearic acid, and the content by weight of second fatty acid, preferably C16 fatty acid, preferably palmitic acid, ranges from 0.8 to 1.8, preferably from 1 to 1.5, preferably from 1.1 to 1.3 and/or the mass ratio between the content by weight of second fatty acid, preferably C16 fatty acid, preferably palmitic acid, and the content by weight of third fatty acid, preferably C14 third fatty acid, preferably myristic acid, ranges from 10 to 20, preferably from 12 to 18, preferably from 14 to 15.5.

Preferably, the content of first fatty acid, preferably C18 fatty acid, preferably stearic acid is between 0.05% and 0.4% by weight in relation to the total weight of the composition, preferably between 0.08% and 0.3% by weight. Preferably, the content of second fatty acid, preferably C16 fatty acid, preferably palmitic acid, is between 0.04% and 0.4% by weight in relation to the total weight of the composition, preferably between 0.06% and 0.3% by weight. Preferably, the content of third fatty acid, preferably C14 third fatty acid, preferably myristic acid, is between 0.001% and 0.03% by weight in relation to the total weight of the composition, preferably between 0.004% and 0.02% by weight.

Advantageously, the third crystallizable fatty substance comprises a mixture of myristic acid, palmitic acid and stearic acid. According to this embodiment, the stearic acid content is greater than or equal to or strictly greater than the palmitic acid content by weight, and the palmitic acid content by weight is greater than or equal to or strictly greater than the myristic acid content by weight.

Preferably, the third crystallizable fatty substance(s) is (are) present with a content of between 0.05% and 2% by weight in relation to the total weight of the composition, preferably between 0.1% and 1% by weight, preferably between 0.15% and 0.8% by weight, advantageously between 0.2% and 0.65% by weight.

Preferably, the total first crystallizable fatty substance content by weight is greater than or equal to, or strictly greater than the second crystallizable fatty substance content by weight. Preferably, the total second crystallizable fatty substance content by weight is greater than or equal to, or strictly greater than the third crystallizable fatty substance content by weight.

Preferably, the mass ratio between the total first crystallizable fatty substance content by weight and the total second crystallizable fatty substance content by weight ranges from 1 to 10, preferably from 1.5 to 8, preferably from 2 to 6.

Preferably, the mass ratio between the total second crystallizable fatty substance content by weight and the total third crystallizable fatty substance content by weight ranges from 1 to 5, preferably from 1.1 to 3.

Preferably, the total crystallizable fatty substance content is between 0.5% and 10% by weight in relation to the total weight of the composition, preferably between 1% and 8% by weight, preferably between 1.5% and 5% by weight, advantageously between 1.8% and 4% by weight.

Preferably, the total clay content by weight is greater than or equal to, or strictly greater than the second crystallizable fatty substance content by weight.

Preferably, the total clay content by weight is greater than or equal to, or strictly greater than the third crystallizable fatty substance content by weight.

### Oil

The composition according to the invention can furthermore comprise at least one oil.

The term oil means a fatty substance that is in liquid form at ambient temperature (20 to 25° C.) and at atmospheric pressure (760 mm of Hg).

By way of oils, mention may be made for example of:
hydrocarbon oils of plant origin, preferably chosen from jojoba oil, castor oil, olive oil, coco oil, DHA algae oil, ximenia oil, pracaxi oil, coriander seed oil, macadamia oil, passionflower oil, argan oil, neem oil, bay seed oil, arnica oil (macerate), sesame oil, sunflower oil, grapeseed oil, grapefruit seed oil, marrow seed oil, perilla oil, avocado oil, broccoli oil, rosa canina oil, apricot (or apricot kernel) oil, baobab oil, rosehip oil, milk thistle oil, Dhatelo oil, apricot kernel oil, flax oil, polymerized flax oil, sweet almond oil, cottonseed oil, arugula oil, soybean oil, rapeseed oil, canola oil, peanut oil, kaya oil, nigella oil, musk rose oil, Acai oil, sea buckthorn oil, calophyllum oil, tamanu oil, camelia oil, copra oil, moringa oil, prickly pear oil, walnut oil, coconut oil, bearberry oil, nutgrass oil, carrot oil, Marula oil, Camelina oil, wheatgerm oil, corn oil, corn seed oil, rice bran oil, alfalfa oil, poppy seed oil, pumpkin oil, marrow oil, hazelnut oil, blackcurrant oil, optionally organic pomegranate oil, evening primrose oil, millet oil, barley oil, quinoa oil, rye oil, safflower oil, candleberry oil, limnanthes oil, black cumin oil, buriti oil, sandal nut oil, Babassu oil, karanja oil, kukui oil, lingonberry seed oil, chia oil, hemp oil, oat oil, Inca Inchi oil, cucumber oil, cranberry oil, fenugreek oil, mustard seed oil, maritime pine seed oil, mango oil, tepezcohuite, amaranth, passionfruit, rice bran, raspberry, poppy oil extract, liquid shea butter fraction, and liquid cocoa butter fraction, squalane, liquid triglycerides of fatty acids including from 4 to 30 carbon atoms, and mixtures thereof;
esters and synthetic esters, in particular fatty acids, such as oils having formulas R1COOR2 and R1OR2 wherein R1 is the residue of a fatty acid or fatty acid or a fatty alcohol including from 8 to 29 carbon atoms, and R2 is a hydrocarbon chain, optionally branched, containing from 3 to 30 carbon atoms, such as for example Purcellin oil, octyl-2-dodecyl stearate, octyl-2-dodecyl erucate, isostearyl isostearate; hydroxylated esters such as isostearyl lactate, octylhydroxystearate, octyldodecyl hydroxystearate, diisostearyl-malate, triisocetyl citrate; heptanoates, octanoates, decanoates of fatty alcohols; polyol esters, such as propylene glycol dioctanoate, neopentyl glycol diheptanoate and diethylene glycol diisononanoate; and pentaerythritol esters such as pentaerythrityl tetraisostearate;
linear or branched hydrocarbons, of mineral or synthetic origin, optionally volatile, and derivatives thereof, such as branched alkanes including from 8 to 18 carbon atoms, for example iso-alkanes (also referred to as isoparaffins) at C8-C18 such as isododecane, isodecane, isohexadecane, such as isoparaffins sold under the trade names Isopar by Exxon Chemical or the oils sold under the trade names Permethyl by Presperse, isohexadecane and isododecane marketed by INEOS; as well as petrolatum oil and hydrogenated polyisobutene such as Parleam® oil; volatile linear alkanes comprising from 7 to 17 carbon atoms such as undecane, tridecane;
fatty alcohols that are liquid at ambient temperature having from 8 to 26 carbon atoms, such as octyldodecanol, 2-butyloctanol, 2-hexyldecanol, 2-undecylpentadecanol or oleic alcohol; and
mixtures thereof.

Mention may furthermore be made of the following oils:
esters obtained from reacting at least one fatty acid including at least 6 carbon atoms, preferably from 6 to 26 carbon atoms and more preferably from 6 to 20 carbon atoms, even more preferably from 6 to 16 carbon atoms and at least one alcohol comprising from 1 to 17 carbon atoms and more preferably from 3 to 15 carbon atoms; mention may in particular be made of isopropyl myristate, isopropyl palmitate, ethyl-2-hexyl caprate/caprylate (or octyl caprate/caprylate), ethyl-2-hexyl palmitate, isostearyl neopentanoate, isononyl isononanoate, ethyl laurate, esters of lactic acid and fatty alcohols comprising 12 or 13 carbon atoms, dicaprylyl carbonate such as that marketed under the trade name CETIOL CC by COGNIS,
fatty acid ethers comprising from 6 to 20 carbon atoms such as dicaprylyl ether (Cetiol OE from Cognis), —glycerol ethers comprising from 6 to 12 carbon atoms such as 2-ethyl hexyl glycerol ether (INCI name: ethylhexylglycerin) such as Sensiva SC 50 from Schulke & Mayr GmbH;
octydodecanol,
alkanes such as those described in patent applications WO 2007/068371, or WO2008/155059 from Cognis (separate alkane mixtures differing by at least one carbon). These alkanes are obtained from fatty alcohols, themselves obtained from copra or palm oil.

As an example of linear alkanes suitable for the invention, mention can be made of n-heptane (C7), n-octane (C8), n-nonane (C9), n-decane (C10), n-undecane (C11), n-dodecane (C12), n-tridecane (C13), n-tetradecane (C14) and mixtures thereof. According to one particular embodiment, the volatile linear alkane is chosen from among n-nonane, n-undecane, n-dodecane, n-tridecane, n-tetradecane and mixes thereof.

According to one preferred embodiment, mention can be made of mixtures of n-undecane (C11) and n-tridecane (C13) obtained in examples 1 and 2 in application WO2008/155059 from Cognis;
polyesters obtained by condensing unsaturated fatty acid dimers and/or trimers and diols such as for example the dilinoleic acid and diol polyesters marketed by Biosynthis under the trade name Viscoplast and in particular the polymer bearing the INCI name dilinoleic acid/propanediol copolymer; and
mixtures thereof.

Preferably, the oil(s) are chosen from hydrocarbon oils of plant origin as listed above, preferably from apricot (or apricot kernel) oil and sunflower (or sunflower seed oil).

Preferably, the oil content is between 0.5% and 5% by weight in relation to the total weight of the composition, preferably between 1% and 4% by weight, advantageously between 1.4% and 3.6% by weight.

Preferably, the mass ratio between the total crystallizable fatty substance content and the total oil content ranges from 0.3 to 4, preferably from 0.4 to 3, preferably from 0.5 to 2.5. Such a ratio makes it possible to optimize the crystallization of the crystallizable fatty substance(s) and the formation of spherulites, which enhances the blurring effect of the composition according to the invention

### Polymer

The composition according to the invention may furthermore comprise at least one polymer. This polymer is for example a thickener and/or a gelling agent and/or an emulsifier.

Examples of polymers include carboxyvinyl polymers such as Carbopols® (Carbomers) and Pemulens such as Pemulen TR1® and Pemulen TR2® (acrylate/C10-C30 alkylacrylate crosspolymer); polyacrylamides such as for example cross-linked copolymers sold under the names Sepigel 305® (C.T.F.A. name: polyacrylamide/C13-14 isoparaffin/Laureth 7) or Simulgel 600® (C.T.F.A. name: acrylamide/sodium acryloyldimethyltaurate copolymer/isohexadecane/polysorbate 80) by Seppic; 2-acrylamido 2-methylpropane sulfonic acid polymers and copolymers, optionally cross-linked and/or neutralized, such as poly(2-acrylamido 2-methylpropane sulfonic acid) marketed by Hoechst under the trade name “Hostacerin AMPS®” (CTFA name: ammonium polyacryloyldimethyl taurate or SIMULGEL 800® marketed by SEPPIC (CTFA name: sodium polyacryolyldimethyl taurate/polysorbate 80/sorbitan oleate); 2-acrylamido 2-methylpropane sulfonic acid and hydroxyethyl acrylate copolymers such as SIMULGEL NS® and SEPINOV EMT 10® marketed by SEPPIC; cellulose derivatives such as hydroxyethylcellulose; natural or synthetic polysaccharides such as starch, cellulose, dextrans, inulin, chitin (in particular chitosan, alginic acid and alginates), plant gums such as xanthan gum or cellulose gum, pectin; water-soluble or water-dispersible silicone derivatives such as acrylic silicones, silicone polyethers and cationic silicones and mixtures thereof.

The polymers are preferably chosen from natural or synthetic polysaccharides, preferably from natural polysaccharides, advantageously from plant gums, alginates and pectin, and mixtures thereof.

The polymers are preferably present in a quantity between 0.01% and 5% by weight, preferably between 0.02% and 2% by weight, advantageously between 0.04% and 0.8% by weight in relation to the total weight of the composition.

The presence of such a polymer further enhances the stability of the composition according to the invention, in particular by limiting the syneresis phenomenon.

### Aqueous Phase

The composition according to the invention comprises a physiologically acceptable aqueous medium.

Preferably, the composition according to the invention comprises an aqueous phase comprising at least water.

The water used can be sterile demineralized water and/or floral water such as rose water, cornflower water, chamomile water or linden water, and/or a spring or natural mineral water, such as for example: Vittel water, water from the basin of Vichy, Uriage water, la Roche Posay water, la Bourboule water, Enghien-les-bains water, Saint Gervais-les-Bains water, Neris-les-Bains water, Allevar-les-Bains water, Digne water, Maizières water, Neyrac-les-Bains water, Lons-le-saunier water, les Eaux Bonnes, Rochefort water, Saint Christau water, Fumades water and Tercis-les-bains water, Avène water.

The aqueous phase can furthermore comprise at least one other organic solvent soluble in water, at 25° C., chosen for example from among:
C1-C4 monoalkanols. The term “C1-C4 monoalkanol” means any saturated, linear or branched alkane compound with 1 to 4 carbon atoms and a single hydroxyl (OH) function. The C1-C4 monoalkanols present in the compositions according to the invention can be chosen from among methanol, ethanol, propanol, isopropanol, butanol or mixtures thereof,
polyols particularly with 2 to 20 carbon atoms, preferably 2 to 6 carbon atoms, such as glycerol, diglycerol, propyleneglycol, isoprene glycol, dipropyleneglycol, butylene glycol, hexylene glycol, 1,3-propanediol, 1,2-octanediol, pentylene glycol, simple sugars, water-soluble polyalkyleneglycols; and
mixtures thereof.

Preferably, the other water-soluble organic solvent(s) are chosen from the list consisting of 1,2-octanediol, glycerol and 1,3-propanediol.

They may be present in concentrations ranging from 0.5% to 50% by weight, more preferably from 1% to 30%, and preferably from 5% to 15% by weight in relation to the total weight of the composition.

### Fatty Phase

Preferably, the composition according to the invention comprises a fatty phase, preferably crystallized. The fatty phase comprises at least one crystallizable fatty substance in crystallized form, and optionally one or more oils, preferably encapsuled in the crystallizable fatty substance in crystallized form.

Preferably, the total fatty phase content is between 0.5% and 10% by weight in relation to the total weight of the composition, preferably between 2% and 8% by weight, preferably between 4% and 7% by weight, advantageously between 5% and 6% by weight.

### Composition

Preferably, the composition according to the invention is substantially free from surfactant. “Substantially free from surfactants” means that the composition according to the invention has a surfactant concentration less than or equal to 2% by weight, with respect to the total weight of the composition, preferably less than or equal to 0.5% by weight, preferably between 0.01% and 2% by weight. The composition is preferably substantially free from non-ionic surfactants, in particular chosen from polyethoxylated, polypropoxylated or polyglycerolated fatty alcohols, polyethoxylated, polypropoxylated or polyglycerolated α-diols, and polyethoxylated, polypropoxylated or polyglycerolated (C1-C20) alkylphenols, the fatty chain comprising, for example, from 8 to 18 carbon atoms, the number of ethylene oxide or propylene oxide groups optionally in particular varying from 1 to 150 and the number of glycerol groups optionally in particular varying from 1 to 30, polyglycerol C6-C30 fatty acid esters, oxyalkylene sugar esters, esters of fatty acid and polyethylene glycol at C6-C30, esters of fatty acid and sorbitan at C6-C30, glycerol (di) esters, glucamine derivatives and mixtures thereof, for example Glyceryl citrate stearate. Preferably, the composition according to the invention is totally free from surfactant, preferably totally free from surfactant is as defined above.

Preferably, the composition according to the invention has a pH between 5 and 12, preferably between 5.5 and 8, preferably between 6 and 7.

Preferably, the composition according to the invention comprises an aqueous gel comprising a crystallized fatty phase dispersion.

Preferably, the composition according to the invention comprises a fatty phase as defined above dispersed in an aqueous phase as defined above.

Preferably, the crystallizable fatty substance(s) in crystallized form (preferably the solid fatty phase) of the composition according to the invention is (are) in the form of substantially spherical particles (in particular in the form of spherulites) of number average diameter between 200 nm and 100 μm, preferably between 1 μm and 50 μm, preferably between 2 μm and 30 μm.

Preferably, the composition comprises at least particles of crystallizable fatty substance in crystallized form (for example spherulites) of a size strictly greater than 2 μm, preferably of a size strictly greater than 3 μm, preferably of a size strictly greater than 4 μm, more preferably of a size strictly greater than 5 μm.

Furthermore, the composition according to the invention may comprise one or more conventional cosmetic additives chosen in particular from softeners; antioxidants; active agents; stabilizers; sequestering agents; anti-foaming agents; hydrating agents; vitamins; perfumes; preservatives; fillers; or any other ingredient customarily used in cosmetics and/or dermatology, in particular to manufacture compositions in the form of aqueous gels.

The expressions “between . . . and . . . ”, “ranging from . . . to . . . ” and “varies from . . . to . . . ” are to be understood to be inclusive of the limits, unless specified otherwise.

Concrete, yet non-limiting, examples, illustrating the invention, will now be provided.

### FIGURES

FIG. 1 consists of photographs taken by microscopy at X40 magnification, with DIC (Differential Interference Contrast) for a) composition C1 according to the invention, b) composition C2 according to the invention, and c) comparative composition C3*.

### EXAMPLES

### Example 1: Preparation of Compositions According to the Invention C1 and C2 and Comparative Compositions C3* and C4*

The compositions according to the invention C1 and C2 and comparative compositions C3* and C4* are prepared with the ingredients mentioned in the table hereinbelow, according to the following protocol:

Preparation of the aqueous phase: dispersion for 10 minutes under rotor stator of hectorite in a portion of the water (40 to 45%) and glycerin heated to 60° C. in C1 C2 C4*/70° C. in C3*. Addition of phytic acid (optional) followed by polymers.

In parallel; preparation of the fatty phase: melt the fatty phase at the melting point of the fatty substances of the composition: 60° C. in C1 C2/70° C. in C3*.

Dispersion of the fatty phase in the aqueous phase under rotor stator followed by cooling with addition of the remaining water and propanediol. At 35° C., add caprylyl glycol, ethanol, benzyl alcohol and/or arginine, according to the compositions.

The quantities of each ingredient are expressed as a mass ratio in relation to the total weight of the composition.

TABLE 1

| | | | | C4* |
|---|---|---|---|---|
| | | | | (Aqueous |
| | C1 | C2 | C3* | phase of C2) |
| | (% w/w) | (% w/w) | (% w/w) | (% w/w) |
| Ingredient | (invention) | (invention) | (comparative) | (comparative) |
| Water | QS 100 | QS 100 | QS 100 | QS 100 |
| Glycerin | 7.00 | 7.00 | 16.00 | 7.423 |
| Propanediol | 4.00 | 4.00 | 5.00 | 4.242 |
| Hectorite | 3.00 | 2.00 | 2.00 | 2.121 |
| Sucrose | — | 0.0020 | — | 0.002 |
| Cellulose gum | 0.05 | — | — | — |
| Sodium alginate | — | 0.50 | — | 0.530 |
| Pectin | — | 0.008 | — | 0.008 |
| Xanthan gum | — | — | 0.10 | — |
| Myristic acid | 0.006 | 0.015 | 0.006 | — |
| Palmitic acid | 0.088 | 0.22 | 0.088 | — |
| Cetearyl alcohol | 3.00 | 1.00 | 3.00 | — |
| Stearic acid | 0.106 | 0.265 | 0.106 | — |
| Myristyl stearate | 0.30 | 0.30 | 0.30 | — |
| Myristyl palmitate | 0.20 | 0.20 | 0.20 | — |
| Glyceryl citrate | — | — | 3.00 | — |
| stearate | | | | |
| Apricot kernel | 1.50 | 3.50 | — | — |
| plant oil | | | | |
| Sunflower seed oil | 0.02 | 0.02 | — | — |
| Caprylic/capric | — | — | 5.30 | — |
| triglycerides | | | | |
| Dicaprylic ether | — | — | 5.9982 | — |
| Octyldodecanol | — | — | 2.70 | — |
| Tocopherol | 0.18 | 0.18 | 0.0018 | — |
| Benzyl alcohol | — | — | 1.00 | — |
| Caprylyl glycol | 0.50 | 0.50 | — | 0.530 |
| Arginine | — | 0.12 | — | 0.127 |
| Phytic acid | 0.0750 | 0.075 | — | 0.080 |
| Denatured alcohol | — | — | 5.586 | — |
| Total FATTY PHASE | 5.40 | 5.70 | 21.70 | 0 |
| Ratio of | 2.43 | 0.57 | 0.26 | — |
| crystallizable fatty | | | | |
| substance/oils | | | | |
| Size of objects | heterogeneous | heterogeneous | homogeneous | — |
| formed by the | 1-30 μm | 1-30 μm | 1-2 μm | |
| fatty phase | | | | |
| (spherulites | | | | |
| and/or droplets) | | | | |
| Presence of particles | yes | yes | no | — |
| (spherulites) of | | | | |
| size > 5 μm | | | | |

Compositions C1 and C2 according to the invention comprise a crystallized fatty phase, comprising at least one crystallizable fatty substance in crystallized form. On the other hand, the comparative composition C3* comprises a non-crystallized fatty phase. It comprises in particular a surfactant (Glyceryl citrate stearate).

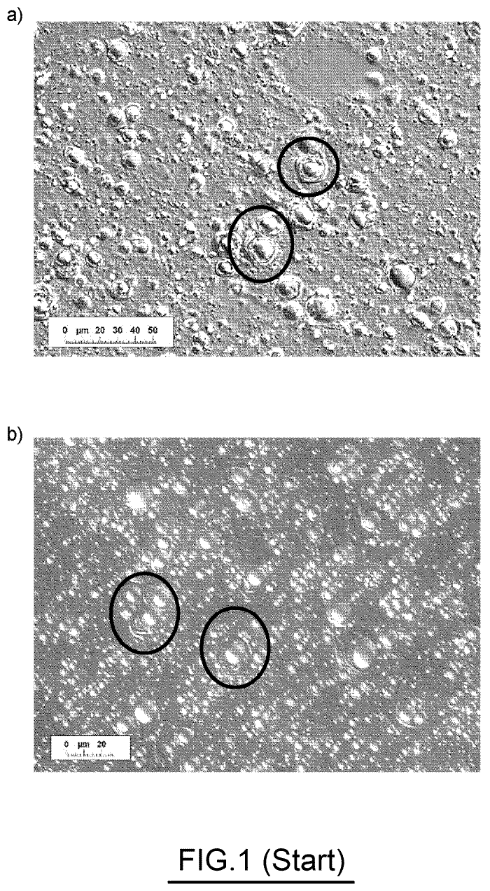

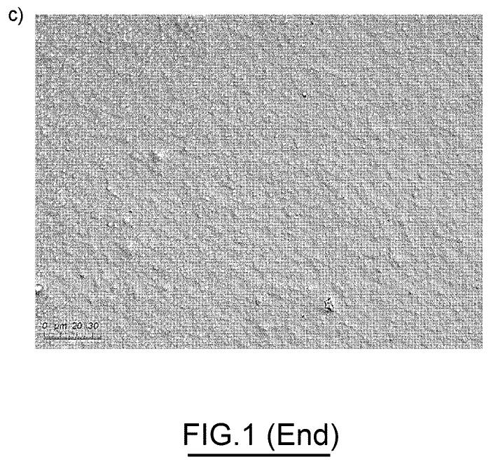

This structural difference is visible in optical microscopy (see FIG. 1). The photos taken in microscopy at X40 magnification, with DIC (Differential Interference Contrast), clearly show the lamellar structures around the globules which identify the fatty acid spherulites in compositions C1 and C2 according to the invention (FIGS. 1a and b). On the other hand, the microscopy of comparative composition C3* does not show these crystalline structures (FIG. 1c).

Composition C4* does not comprise a fatty phase.

### Example 2: Evaluation of Compositions According to the Invention C1 and C2 and Comparative Composition C3*

Compositions according to the invention C1 and C2 and comparative composition C3* of example 1 were evaluated in vitro on the following features:

### Stability

The compositions are studied for stability in glass pill bottles, at ambient temperature (AT), at 45° C. and 4° C. for 2 months. The appearance, color, viscosity, microscopy and pH are checked.

### Measurement of Blurring Effect of Haze

A film of each composition is spread on a PET sheet using a BYK automatic spreader-film thickness 25 μm-air-drying for one hour-Measurement of blurring with a HAZEGARD (Haze “H” and transmittance “T”)-3 measurements per film/average on 2 films.

### Gloss Measurement

A film of each composition is spread on a BYK 2814 gloss contrast chart using a BYK automatic spreader—film thickness 25 μm—air-drying for one hour—gloss measurement using a gloss-meter (BYK) at 60°.

### Sweat and Sebum Resistance Measurement

A water/sebum mixture approximating the hydrolipid film of the skin is modeled by preparing a Vichy spring water/oleic acid/Oleth-10 mixture (respective weight ratio: 79:20:1).

For each composition, a 100 μm thick film is spread on an ERICHSEN gloss contrast chart using a BYK automatic spreader.

The film is dried for 24 h in an oven at 37° C.

The gloss of the film after drying is measured using the gloss-meter (BYK) at 60°.

The Vichy spring water/oleic acid/Oleth-10 mixture is sprayed onto the film once said mixture has returned to ambient temperature. The gloss of the film is measured using the gloss-meter (BYK) at 60° after 6 min after spraying, then the film is sprayed again, and the gloss is measured again 6 min after this second spraying, then the film is sprayed a third time, and the gloss is measured again 6 min after this third spraying. The values measured are referred to as gloss units GU. A performance index is calculated, which makes it possible to quantify the performance of the compositions.

For comparison, a negative reference (Gel with amino-propane-sulfonic acid—AMPS—no filler, index=0) and a positive reference (AMPS gel with 2% aerogel, index=100) were chosen. This, for each composition, the index is calculated to position it on a scale of 0 to 100. The index is calculated according to the following formula:

$$Index ( Compo Cx , T 6 min ) = \frac{UB ( Compo Cx , T 6 min ) \text{?} UB ( \text{?} , T 6 min )}{UB ( \text{?} positive , T 6 min ) - UB ( \text{?} , T 6 min )}$$

$$\text{?} \text{indicates text missing or illegible when filed}$$

If the index is greater than 75, the composition is considered to be highly effective.

If the index is between and 60 and 75, the composition is considered to be effective.

### Results

TABLE 2

| | C1 | C2 | C3* | C4* |
|---|---|---|---|---|
| Stability | Stability | Stability | Stability | Syneresis after a |
| | conforming at T2 | conforming at T2 | conforming at T2 | few days at AT |
| | months AT and | months AT; very | months AT and | |
| | 45° C. | slight syneresis | 45° C. | |
| | | (some drops) at | | |
| | | T2M 45° C. | | |
| Blurring effect | 46.5 +/− 1.01 | 53.9 +/− 1.3 | 10.9 +/− 0.7 | 18 +/− 0.07 |
| | (T > 90) | (T > 90) | (T > 90) | (T > 90) |
| Gloss | 4.7 +/− 0.07 | 4.9 +/− 0.01 | 40.1 +/− 2.7 | 23.3 +/− 0.28 |
| Sweat and | T6 min = | T6 min = | T 6 min = | — |
| sebum | 81.8 +/− 0.51 | 87.2 +/− 0.52 | 71.4 +/− 0.44 | |
| resistance | T18 min = | T18 min = | T 18 min = | |
| | 86.7 +/− 1.33 | 89.8 +/− 1.36 | 82 +/− 1.26 | |

For compositions C1, C2 and C3*, the stability is significant enhanced compared to formulas with no fatty phase (C4*).

Regarding the blurring effect, substantial blurring effects are observed using compositions C1 and C2 according to the invention, wherein the fatty phase is recrystallized in spherulite form. On the other hand, the blurring effects are poor with comparative compositions C3* and C4*.

Furthermore, the mattness is significantly increased with the presence of the crystallized fatty phase (compositions C1 and C2).

In conclusion, the presence of hectorite makes it possible to obtain a very good sebum and sweat absorption effectiveness, and the recrystallized fatty phase makes it possible to furthermore provide blurring, mattness and therefore imperfection masking effects.

In vivo tests performed make it possible to confirm these results and demonstrate that the sensory properties correspond to a cosmetic application (example 3).

### Example 3: In Vivo Evaluation of Compositions According to the Invention and Comparative Compositions

Composition C1 according to the invention from example 1 was compared in vivo with a simplex hectorite gel (composition C5*), comprising 2% by weight of hectorite, 0.5% by weight of denatured alcohol and the remainder water, and with a reference composition C6* corresponding to a hydrating and mattifying treatment (GARNIER Pure active sensitive—Anti-imperfection hydrating treatment).

None of these compositions comprises crystallizable fatty substance in crystallized form.

The in vivo test followed is as follows:

The steam bath test is performed on volunteers. A gloss measurement is performed prior to application of the product on 4 areas of the face measuring 4 cm×3.5 cm (2 on the forehead and 2 on the cheeks) using a light-Cam (T0). After applying the formula and drying for 10 min, another measurement is made (Timm). The volunteers then spend 2H in a steam bath to encourage sweating, then another measurement is made (T2H).

The results obtained are compiled in the following table:

TABLE 5

| | C1 | C5* | C6* |
|---|---|---|---|
| Timm-T | −11.12 +/− 2.43 | −14.53 +/− 2.58 | −4.68 +/− 1.29 |
| T2H-T0 | −6.84 +/− 2.46 | −6.29 +/− 2.25 | 2.45 +/− 1.72 |

Composition C1 according to the invention and simplex gel C5* show a significant effectiveness on sweat and sebum absorption, superior to composition C6*. On the other hand, simplex gel C5* exhibits substantial syneresis after a few days at ambient temperature. This demonstrates that the compositions according to the invention comprising a clay chosen from phyllosilicates having a 2:1 sheet structure and a crystallized fatty phase make it possible to obtain a stable composition providing long-term mattness.

1. A composition, comprising, in a physiologically acceptable aqueous medium:
at least one clay chosen from phyllosilicates having a 2:1 sheet structure, and
at least one crystallizable fatty substance in crystallized form.

2. The composition according to claim 1, wherein it is substantially free from surfactant.

3. The composition according to claim 1, wherein the crystallizable fatty substance comprises at least a first crystallizable fatty substance, at least a second crystallizable fatty substance and at least a third crystallizable fatty substance, the first, second and third crystallizable fatty substances being different from each other, and the first, second and third crystallizable fatty substances being each in crystallized form.

4. The composition according to claim 3, wherein the first crystallizable fatty substance is chosen from saturated or unsaturated C12-C24 fatty chain alcohols.

5. The composition according to claim 3, wherein the second crystallizable fatty substance is chosen from saturated or unsaturated fatty chain esters.

6. The composition according to claim 3, wherein the third crystallizable fatty substance is chosen from saturated or unsaturated C10-C24 fatty acids and mixtures thereof.

7. The composition according to claim 1, wherein the total crystallizable fatty substance content is between 0.5% and 10% by weight in relation to the total weight of the composition.

8. The composition according to claim 1, wherein the clay is chosen from smectites.

9. The composition according to claim 1, wherein the clay is present with a content between 0.5% and 8% by weight in relation to the total weight of the composition.

10. The composition according to claim 1, further comprising at least one oil.

11. The composition according to claim 10, wherein the mass ratio between the total crystallizable fatty substance content and the total oil content ranges from 0.3 to 4.

12. The composition according to claim 1, wherein the crystallizable fatty substance(s) in crystallized form is (are) in the form of substantially spherical particles of number average diameter between 200 nm and 100 μm.

13. (canceled)

14. A cosmetic method for regulating excess sebum of the skin and/or for mattifying the skin and/or blurring skin imperfections, comprising applying on the skin at least one cosmetic composition according to claim 1.

15. A method for enhancing the mattifying and/or blurring effect of a cosmetic composition comprising at least one clay chosen from phyllosilicates having a 2:1 sheet structure, comprising the addition of at least one crystallizable fatty substance in crystallized form in said composition.

16. The composition according to claim 3, wherein the first crystallizable fatty substance is chosen from stearyl alcohol, myristyl alcohol and cetyl alcohol.

17. The composition according to claim 3, wherein the second crystallizable fatty substance comprises a mixture of at least two esters chosen from esters of palmityl, cetyl or myristyl alcohols, and palmitic, myristic or stearic acids.

18. The composition according to claim 3, wherein the third crystallizable fatty substance is a mixture of myristic acid, palmitic acid and stearic acid.

19. The composition according to claim 1, wherein the clay is chosen from the group consisting of hectorite, stevensite, saponite, sauconite, montmorillonite, beidellite, nontronite, ferrosaponite and saliotite.

20. The composition according to claim 1, wherein the clay is non-modified.

21. The composition according to claim 3, wherein the first crystallizable fatty substance is chosen from stearyl alcohol, myristyl alcohol and cetyl alcohol; the second crystallizable fatty substance comprises a mixture of at least two esters chosen from esters of palmityl, cetyl or myristyl alcohols, and palmitic, myristic or stearic acids; the third crystallizable fatty substance is a mixture of myristic acid, palmitic acid and stearic acid; and the clay is chosen from the group consisting of hectorite, stevensite, saponite, sauconite, montmorillonite, beidellite, nontronite, ferrosaponite and saliotite.
